(19) United States
(12) Patent Application Publication
(10) Pub. No.: US 2017/0247275 A1
(43) Pub. Date: Aug. 31, 2017

(54) SENSING METHODS AND SYSTEMS

(71) Applicant: Bactest Limited, Cambridge (GB)

(72) Inventors: Derek Price, Cambridge (GB); Toby William Rickett, Cambridge (GB)

(73) Assignee: Bactest Limited, Cambridge (GB)

(21) Appl. No.: 15/516,383

(22) PCT Filed: Sep. 30, 2015
(86) PCT No.: PCT/GB2015/052862

§ 371 Date: Mar. 31, 2017

(30) Foreign Application Priority Data
Oct. 1, 2014 (GB) 1417309.0

(51) Int. Cl. C02F 3/00; C02F 3/12; G01N 33/18

(52) U.S. Cl. CPC C02F 3/006; G01N 33/1806; C02F 3/12; G01N 33/1866; G01N 27/22

(57) ABSTRACT

We describe a method of determining a food-to-biomass ratio in an aqueous fluid the method comprising: providing an aqueous fluid comprising viable biomass and food for said biomass, and wherein there is insufficient available food to sustain all said viable biomass; using a sensor (for example a respirometer or a sensor for sensing an amount of ammonia, ammonium, nitrates or nitrites) to determine an amount of food in said aqueous fluid available to said biomass; determining a measure of viable biomass in said aqueous fluid by measuring polarisability of viable biomass cells in an AC electric field; and determining a food-to-biomass ratio from said amount of food and said measure of viable biomass.

### FIELD OF THE INVENTION

This invention relates to sensing methods and systems, in particular for monitoring living organisms in water, in some preferred applications for monitoring activated sludge in sewage treatment plants.

### BACKGROUND TO THE INVENTION

Waste water treatment accounts for a surprisingly large proportion of the total UK energy supply, by some estimates up to 2%. The majority of this energy goes to aeration of the biological floc in a treatment plant. By way of an example, a sewage treatment plant might have perhaps twenty 100 KW motors running continuously to provide aeration for the activated (bacteria-containing) sludge. It is likely that a much lower degree of aeration would suffice, but understandably a plant manager will err on the side of caution. The process of keeping a waste water treatment plant running satisfactorily is relatively poorly understood and generally not closely controlled.

In the main sight and smell are used by experienced managers to control a plant (when operating properly, the smell is not unpleasant), supplemented by occasional tests. In the UK typically the BOD5 test (biochemical oxygen demand 5 day test) is used which, as the name implies, incubates a field sample over five days to characterise the sample by its oxygen use. We have previously described improved techniques for BOD5-type testing, in WO2014/029976. Sometimes probes such as an oxygen or ammonia probe are also employed although in practice these do not work well and often fail or go out of calibration.

Better understanding and control of the process could enable substantial energy savings by reducing the degree of aeration to just that which is necessary. However it is difficult to obtain accurate measurements of the status of a plant, and in particular of the status of the activated sludge. This is in part because the aqueous sludge is not a pure liquid but includes insoluble particles, household/industrial rubbish and the like. General background prior art relating to monitoring biomass by measuring electrical capacitance/permittivity can be found in U.S. Pat. No. 5,182,193 and in Pajoum-Shariati et al, “Dielectric monitoring and respirometric activity of a high cell density activated sludge”, Environ Technol, 2014 January-February, 35(1-4): 425-31.

We have previously described techniques for closed-loop control of a waste water treatment plant, in WO2014/027183. These comprise providing a fluid sample to a sealed chamber such that the sample incompletely fills the chamber leaving a head-space; incubating said fluid sample in the chamber; determining a change in pressure in the head-space during the incubation; and controlling a degree of aeration of the waste water treatment plant responsive to the change in pressure. In preferred implementations two fluid samples are obtained, one for determining a parameter representing a level of food in the influent, another for determining a quantity of living biological material (biomass) in the plant. The former sample may be obtained, for example, from the influent to the plant; the latter from the RAS (returned activated sludge) corresponding parameters may be obtained from respective changes in headspace pressure when incubating these two fluid samples. These parameters represent, respectively levels of food and biomass in the plant. The degree of aeration may then be controlled responsive to a combination of the parameters, in particular responsive to a ratio of food to biomass. The particular degree of aeration/control may be determined on a plant-by-plant basis: typically plants have their own individual characteristics and needs and the control over the aeration equipment may be adapted accordingly.

Whilst these techniques are useful there is a need for improvement. Thus there are many plants in which the influent is combined with the RAS to form a combined flow of Activated Sludge (AS). It would be desirable to be able to make a measurement of food-to-biomass ratio on such an Activated Sludge sample.

### SUMMARY OF THE INVENTION

According to the present invention there is therefore provided a method of determining a food-to-biomass ratio in an aqueous fluid the method comprising: providing an aqueous fluid comprising viable biomass and food for said biomass, and wherein there is insufficient available food to sustain all said viable biomass; using a sensor to determine an amount of food in said aqueous fluid available to said biomass; determining a measure of viable biomass in said aqueous fluid by measuring polarisability of viable biomass cells in an AC electric field; and determining a food-to-biomass ratio from said amount of food and said measure of viable biomass.

The inventors have recognised that a combination of a sensor, for example a respirometer or an ammonium, ammonia, nitrite or nitrate sensor, and a capacitance-based measure of viable biomass may be employed for determining a food-to-biomass ratio, in particular for the AS (activated sludge) of a sewage treatment plant. In embodiments the measurement may be made on a sample from a combined flow of RAS and influent, as described in the introduction. In embodiments the measurement may be made on a single sample of Activated Sludge (AS)—both the respirometer measurement and the capacitance-based measurement may optionally be made on the same Activated Sludge (AS) sample.

In embodiments the aqueous fluid may be a combined flow of Activated Sludge (AS), combined from a first, influent flow and a second, RAS flow. In such a case the measurement of polarisability (capacitance measurement) may be made upstream of where the flows combine (whilst the respirometer measurement may be on a sample of the combined flow of Activated Sludge).

A respirometer, preferably of a type we have previously described (for example in U.S. Pat. No. 8,389,274), responds to the activity of live biomass (for example bacteria, protozoa and like) in the aqueous fluid, in particular AS. Similarly a capacitance measurement can be used to measure viable mass within an aqueous fluid because, it appears, that viable cells are polarizable whereas dead cells are less polarizable. It might appear that these two techniques are measuring essentially the same thing, but the inventors have recognised that they can be combined to determine a food-to-biomass ratio within the aqueous fluid (AS) by selecting a sample, for example from an appropriate location in the treatment plant, in which there is insufficient available food to sustain the viable biomass present. In this case the respirometer, which measures the activity of the live biomass, effectively measures a quantity or concentration of food within the sample, whilst the capacitance/polarisability-based measure of viable biomass measures the total viable biomass in the sample. Thus by a comparison between these two measurements, typically by taking a ratio of the measurements but in principle in some other manner, a food-to-biomass ratio in the sample may be determined.

In some preferred embodiments the respirometer comprises a sealed chamber as described in the introduction above with reference to WO'183; preferably a second, control chamber is also provided. In some preferred implementations the respirometer, whether comprising one or two chambers, is a floating respirometer, in particular comprising a buoyancy device to support the respirometer chamber so that when the respirometer is floating in the aqueous fluid the chamber is partially filled with the fluid to define an enclosed head space above the fluid in which a gas (pressure) sensor may then be located.

In embodiments the system to measure the polarisability of viable biomass cells preferably comprises a probe bearing one or a pair of electrodes (depending upon whether or not capacitance to ground is measured), coupled to a capacitance measuring circuit to provide an output which is responsive to the real and/or complex electrical permittivity of the sample. Preferably the measurement is made at an AC frequency in the range 1 KHz-100 MHz, more preferably in the range 10KHz-10 MHz, for example at around 1 MHz. The capacitance, or a value dependent upon the capacitance, may be used directly as a measure of the viable biomass. However in some preferred embodiments the measured signal is scaled and/or offset to determine a value representing a quantity, more particularly concentration or density, of viable biomass in the aqueous fluid. In embodiments the offset applied may represent a background level of capacitance, for example between the probe and the surrounding environment. Optionally where the probe is used in-situ in a waste water treatment plant a Faraday cage may be placed around the probe, to allow the sampled fluid to flow past the probe but providing a defined background capacitance—but in practice this has not been found necessary.

The data derived from the respirometer and polarisability (electrical permittivity) measurements is a measure of food and viable biomass respectively, but it may not correspond directly to these quantities. Thus in embodiments one or both of the signals derived from these systems may be scaled and/or offset prior to determining the food-to-biomass ratio.

In some preferred embodiments of the method an in-situ measurement is made of the food-to-biomass ratio of AS in a sewage treatment plant, that is without needing to remove a sample from the plant. Preferably the determined food to biomass ratio is then employed for real time control of a degree of aeration of the plant responsive to the determined ratio. Such an approach can potentially provide substantial power savings when running the plant.

In embodiments of the method measurements may be made at a plurality of different AC frequencies or over a substantially continuous range of frequencies. It appears that it may be possible to distinguish between different types or classes of biomass based on the measured capacitance/permittivity at two or more different frequencies. For example, different types of biomass are generally found at different locations within a treatment plant: towards the inlet or towards an upstream direction typically carbonaceous biomass dominates, whereas later different cell types are found and typically nitrogenous biomass (bacteria) dominate.

The terms carbonaceous and nitrogenous are generally used in the art—broadly speaking the biochemical oxygen demand for the degradation of organic material has a carbonaceous oxygen demand component and a nitrogenous oxygen demand component. The carbonaceous demand depends upon the concentration of biomass (organisms) which degrades carbon constituents in the fluid, and the nitrogenous demand depends upon the concentration of biomass (organisms) which metabolise nitrogen (sometimes referred to as nitrifying organisms, organisms that use oxygen to oxidise reduced forms of nitrogen).

In a treatment plant typically carbonaceous organisms dominate towards the inlet, and nitrogenous organisms dominate towards the outlet or back end of the plant. It is desirable to be able to monitor both these populations of organisms, which have different contributions to the biochemical oxygen demand. Embodiments of the method employ one or more probes at two or more different locations within the fluid flow in the plant, to measure the food-to-biomass ratio at these different locations, and more particularly, to distinguish between these two populations. In principle this technique may be extended to distinguish between more than two types of classes of organism. Some embodiments of the method may distinguish between these different populations based on different locations within the plant (ie a non-selective measuring system may be employed at different locations together with an assumption or empirical determination as to which types/classes of organism dominate at the different locations). Additionally or alternatively however, a measurement at one or more locations may be arranged to preferentially respond either to carbonaceous or to nitrogenous viable biomass (cells). This may be achieved by selecting one or more frequencies of operation of the AC electrical measurement, as described in more detail later. Preferably (although not essentially), the system is calibrated in advance so as to be able to distinguish between the different types/classes (eg species) of organism based on AC frequency. As described later a selective response may be achieved by selecting a frequency of AC signal to apply to the electrode(s), but preferably measurements at two different frequencies are compared to distinguish types/classes of organism or are processed to provide a selective response to a particular type/class (eg species) of organism.

Thus in one approach measurements of capacitance/permittivity are made at two different frequencies to, in effect, define a gradient of a line between the values at these two frequencies. This can then be used to differentiate between different populations of organisms such as carbonaceous and nitrogenous.

In more detail, the capacitance/permittivity response as a function of frequency is typically an S-shaped curve (on a linear, or preferably a log frequency scale), for example over a range (in MHz) of 0.01/0.1, to 1.0, to 10/100. Broadly speaking the capacitance/permittivity is relatively higher at low frequencies and lower at high frequencies, so that the S-shaped curve drops to the right along an axis of increasing frequency. It appears that the steepness of the slope or rapidity of the drop depends upon the homogeneity of the sample—the slope appears to be steep for a homogeneous sample but more gradual for a mixed sample. It is postulated that the more gradual slope observed with samples containing a mixture of different types of organism results from the overlap of a set of different curves with high-low capacitance/permittivity transitions at different frequencies. A transition frequency may be defined as the frequency at which the capacitance/permittivity is midway between its low frequency and high frequency values. It is further observed that for a mixed sample the slope of the S-curve is not smooth but exhibits features such as localised bumps, and it is postulated that these are related to the different types/classes of organisms present within the mixed sample.

Thus the invention further contemplates a method of distinguishing between types or classes of organism based on the capacitance/permittivity response of the sample at one or more frequencies or over a range of frequencies. For example embodiments of the method may identify one more features on a curve defined by capacitance/permittivity measurements over a frequency range, to qualitatively identify the presence of two or more different organism types and/or to identify the presence or absence of a particular type or class of organism.

The skilled person will appreciate that where a calibration curve has determined the one or more locations of one or more features which preferentially respond to one or more different types or classes of organism, then one or more measurements may be made selectively at the relevant one or more frequencies to selectively identify the presence of one or more different types of class of organism. Thus although some embodiments of the method may distinguish between types/classes of organism by making measurements at a plurality of different frequencies, it is nonetheless possible to arrange for a measurement to be selectively responsible to a particular type or class of organism by selecting, in principle, just a single frequency of operation.

One problem that can arise in the measurement of viable biomass in an aqueous fluid, particularly (R)AS, is that salts may be present in solution. This can affect the accuracy of the viable biomass measurement. In some preferred embodiments, therefore, the method measures an electrical conductivity (or imaginary component of the permittivity or loss angle), to determine a value dependent upon the conductivity of the aqueous fluid/(R)AS, more particularly salt content of the aqueous fluid/(R)AS. In embodiments the method may then employ this value to adjust or compensate the measure of viable biomass in the sample derived from the capacitance/real component of the permittivity, again for example according to a predetermined calibration curve.

In embodiments of the method, a rate of growth of said viable biomass may be determined, by determining a plurality of measures of viable biomass in said aqueous fluid over a first period of time, and calculating a rate of change of said measure of viable biomass over said first period, said rate of change of said measures of viable biomass being indicative of a rate of growth of said viable biomass.

The method of determining a rate of growth of the viable biomass may be used in conjunction with the above methods of determining a food-to-biomass ratio in the aqueous fluid. Alternatively, the method of determining the rate of growth of the viable biomass may be operated in isolation, that is, the method of determining a rate of growth of viable biomass in an aqueous fluid the method may comprise: providing an aqueous fluid comprising viable biomass and food for said biomass, and wherein there is insufficient available food to sustain all said viable biomass; determining a plurality of measures of viable biomass in said aqueous fluid over a first period of time by measuring polarisability of viable biomass cells in an AC electric field; and calculating a rate of change of said measure of viable biomass over said first period of time, said rate of change of said measures of viable biomass being indicative of a rate of growth of said viable biomass.

In either of the above methods for determining the rate of growth of the viable biomass, the method may comprise differentiating between different cell types within said biomass such that said plurality of measures of viable biomass comprise selective measures of a selected said cell type, and wherein said determined rate of growth of said viable biomass comprises selective rate of growth of viable biomass for a selected subset of said viable biomass in said aqueous fluid.

Furthermore, either of the above methods, may be used to control a waste water treatment plant. That is, a degree of aeration and/or a volume of food of said plant may be controlled responsive to said determined rate of growth of said viable biomass.

In further embodiments, the above methods may comprise determining a rate of conversion of food to viable biomass by: determining a plurality of measures of an amount of food in said aqueous fluid available to said biomass over a second period indicative of a rate of change of amount of food over said second period of time; and determining a plurality of measures of viable biomass in said aqueous fluid over said second period of time indicative of a rate of change of viable biomass over said second period; and calculating a rate of conversion of food to viable biomass by comparing the rate of change of food and rate of change of biomass over said second period of time.

The method of determining a rate of conversion of food to viable biomass may be used in conjunction with the above methods of determining a food-to-biomass ratio in the aqueous fluid and/or the methods of determining the rate of growth of the viable biomass. Alternatively, the method of determining a rate of conversion of food to viable biomass may be operated in isolation, that is, the method of determining a rate of conversion of food to viable biomass in an aqueous fluid may comprise: providing an aqueous fluid comprising viable biomass and food for said biomass, and wherein there is insufficient available food to sustain all said viable biomass; using a sensor to determine a plurality of measures of an amount of food in said aqueous fluid available to said biomass over a second period of time, said plurality of measures of an amount of food indicative of a rate of change of amount of food over said second period of time; determining a plurality of measures of viable biomass in said aqueous fluid over said second period of time by measuring polarisability of viable biomass cells in an AC electric field, said plurality of measures of viable biomass indicative of a rate of change of viable biomass over said second period of time; and calculating a rate of conversion of food to viable biomass by comparing the rate of change of food and rate of change of biomass over said second period of time.

In either of the above methods for determining a rate of conversion of food to viable biomass, the method may further comprise differentiating between different cell types within said biomass such that said plurality of measures of viable biomass comprise selective measures of a selected said cell type, and wherein said determined rate of change of said viable biomass comprises selective rate of change of viable biomass for a selected subset of said viable biomass in said aqueous fluid.

Furthermore, either of the above methods, may be used to control a waste water treatment plant. That is, a degree of aeration and/or a volume of food of said plant may be controlled responsive to said determined rate of conversion of food to viable biomass.

Such additional measures, that is the rate of growth of viable biomass and the rate of conversion of food to viable biomass provide other measures of the health and growth of the biomass under given conditions within a plant, and give additional parameters on which control of a waste water treatment plant may be based in order to improve its efficiency.

Aspects and embodiments of the above described methods may be implemented in a system for measuring a food-to-biomass ratio and/or into a control system for a waste water treatment plant, in particular for controlling aeration of the plant. This may be achieved by providing a respirometer and capacity/permittivity measuring apparatus, and coupling each of these, for example, to a general purpose processor or to dedicated microcontroller, operating under control of program code to combine the data from these two systems to determine a food-to-biomass ratio as previously described.

Thus the invention also provides system for determining a food-to-biomass ratio in an aqueous fluid, the system comprising: a respirometer to determine a level of respiration of biomass within said aqueous fluid, wherein said level of respiration represents an amount of food in said aqueous fluid available to said biomass; cell polarisability measuring apparatus to determine a measure of viable biomass in said aqueous fluid by measuring polarisability of viable biomass cells in an AC electric field; and a system to determine a food-to-biomass ratio from said level of respiration and said measure of viable biomass.

The invention further provides processor control code to implement the above-described systems and methods, for example on a general purpose computer system or on a digital signal processor (DSP). The code is provided on a non-transitory physical data carrier such as a disk, CD- or DVD-ROM, programmed memory such as non-volatile memory (eg Flash) or read-only memory (Firmware). Code (and/or data) to implement embodiments of the invention may comprise source, object or executable code in a conventional programming language (interpreted or compiled) such as C, or assembly code, or code for a hardware description language. As the skilled person will appreciate such code and/or data may be distributed between a plurality of coupled components in communication with one another.

In a further related aspect the invention provides system for differentiating between, or providing a selective response to, different types or classes of viable biomass in activated sludge (AS), the system comprising: a probe for electrically probing said activated sludge (AS); and cell polarisability measuring apparatus, couplable to said probe, to determine a measure of viable biomass in said aqueous fluid by measuring polarisability of viable biomass cells in an AC electric field; wherein said apparatus is configured to differentiate between said different types or classes of viable biomass organisms by operating at one or more selected frequencies or frequency ranges of said AC electric field.

The activated sludge (AS) may be returned activated sludge (RAS). Preferred embodiments of this system are configured, for example using suitable program code, to implement the previously described methods for achieving a selective response to a particular type/class of organism and/or for differentiating between different types/classes of organism.

As previously mentioned, some particularly preferred applications of the techniques we describe are used to control a degree of aeration of an activated sludge vessel of the plant, either manually or automatically. The skilled person will appreciate that it is not necessary to know an absolute food-to-biomass ratio value—a signal indicating a relative increase or decrease of this value can be employed to correspondingly increase or decrease a degree of aeration of the plant. In more sophisticated approaches, a rate of change of this value may additionally or alternatively be employed for controlling the degree of aeration.

Although the embodiments of the method/system are particularly useful for monitoring an activated sludge vessel, it will be appreciated that there are other applications and, in general, the method/system may be employed to monitor and/or control living organisms in any water-based production or processing stage of an industrial plant, for example for monitoring/controlling a fermentation process. Generally, therefore, embodiments of the method/system for automatic monitoring/control of a plant, more particularly aeration monitoring or control, provide or output a signal for example on a screen or printout or on a wired or wireless connection, to a user for manual adjustment/control of the aeration system and/or to a controller for controlling an aeration system for the plant.

As previously mentioned multiple measurements may be made at intervals along the length of an activated sludge vessel in a direction of flow of liquid through the vessel. This is because the conditions change with distance along the flow direction in a sludge vessel (where the liquid may take several hours to transit), for example using more carbon and oxygen at the start and more nitrogen at the end. Thus a potentially more accurate determination of the operating condition of a plant may be achieved using a set of sensors and, optionally, the degree of aeration at different locations within an activated sludge vessel may be different depending upon the locally determined conditions of operation as established local to a respective set of measurements.

### BRIEF DESCRIPTION OF THE DRAWINGS

These and other aspects of the invention will now be further described, by way of example only, with reference to the accompanying figures in which:

FIGS. 1a and 1b show, respectively, a high level schematic diagram of a waste water treatment plant, and schematic block diagram of a control system for closed-loop control of a waste water treatment plant according to an embodiment of the invention;

FIGS. 2a and 2b show a culture vessel which may be adapted for use in embodiments of the invention, showing the vessel under, respectively, normal atmospheric pressure and reduced pressure;

FIG. 3 shows the variation of pressure with time when incubating influent over a period of hours;

FIG. 4 shows a model of a sewage treatment plant usable for calculating a food:biomass ratio;

FIGS. 5a and 5b, show, respectively, growth of biomass in four zones of an activated sludge lane, and an example of an organism growth pattern;

FIG. 6 shows a sewage treatment plant control system according to an embodiment of the invention;

FIG. 7 shows electrical permittivity measurements as a function of frequency in four different zones along an activated sludge (AS) lane of a waste water treatment plant; and

FIGS. 8a and 8b illustrate the use of food-to-biomass sensing systems in a waste water treatment plant.

### DETAILED DESCRIPTION OF PREFERRED EMBODIMENTS

Activated Sludge Monitoring

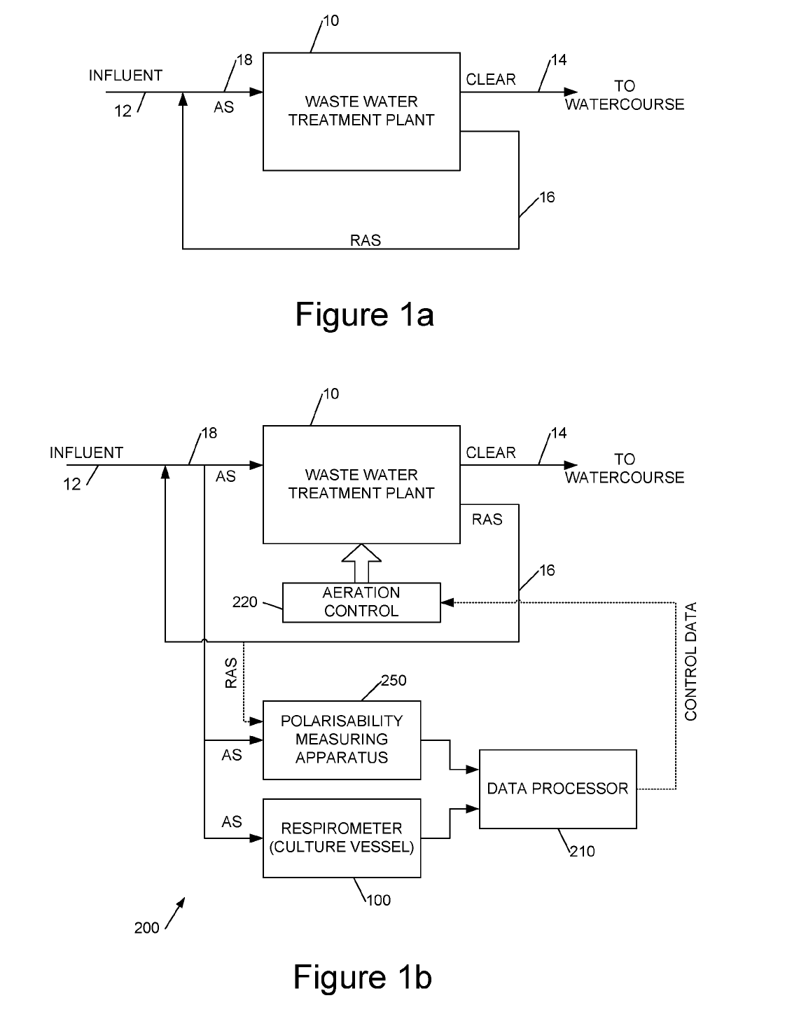

FIG. 1a shows, at a high level, a schematic diagram of the operation of a waste water treatment plant 10. Thus the plant accepts influent 12, fluid from which the solids have been substantially removed, containing a high level of ‘food’ for bacteria, protozoans rotifers, fungi and the like (biomass') and having a high biochemical oxygen demand (BOD). The output from the plant has two components, a clear component 14 which may be provided to a water course and a biological component 16 comprising living biological material referred to as returned activated sludge (RAS), typically at around 60% concentration. The RAS is provided back to the input side of the plant to help maintain the eco system. Often the RAS lane 16 joins the influent lane 12 to form a combined, activated sludge (AS) lane 18.

FIG. 1b shows a block diagram of a closed loop based water treatment control system 200 to implement real time closed loop control of a sewage treatment plant based on a combination of a respirometer and cell polarisability measuring apparatus. The respirometer may be configured to perform a pressure and/or composition measurement of the gases in the headspace of a closed vessel/sealed chamber, as described later, or may operate in some other manner, for example using an oxygen electrode to measure respiration.

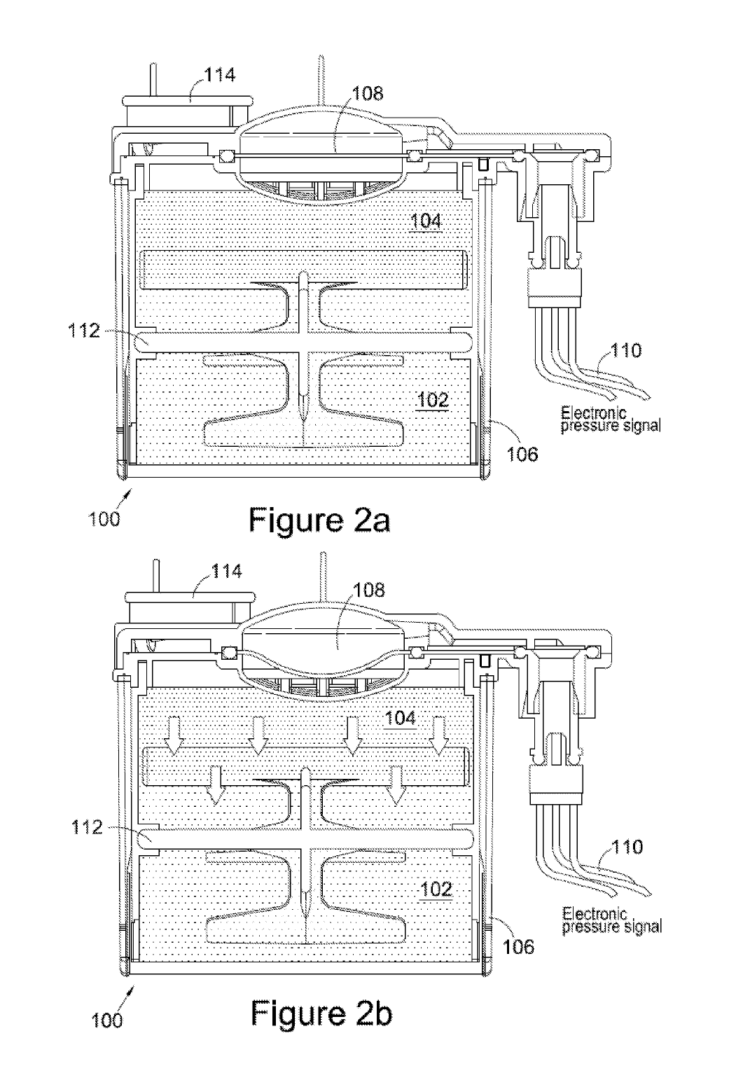

In an example implementation a culture vessel as described with reference to FIG. 2, below, is used as a respirometer 100 to measure the quantity of food in the activated sludge lane 18 (where there is an excess of biomass). The respirometer 100 responds to overall changes in gas pressure/composition within the respirometer. The quantity of biomass is also measured in the activated sludge lane 18, by cell polarisability measuring apparatus 250, although it may alternatively be measured in the RAS lane 16. In some embodiments the respirometer and cell polarisability measuring apparatus may be combined into a single unit—for example a floating respirometer may be combined with a probe bearing one or more electrodes for making a capacitance measurement for the apparatus. In other embodiments they may be separate modules. For example the respirometer may be a device as shown in FIG. 2 and the cell polarisability measuring apparatus may comprise a separate probe into the activate sludge.

The outputs from respirometer 100 and apparatus 250 are each provided to a data processor 210, for example a general purpose computer under software control. Thus measures of both food and biomass, at one or more locations within the plant, are provided to the data processor 210. The data processor may output one or more parameters indicating the food-to-biomass ratio and/or BOD (biochemical oxygen demand) at one or more locations in the system, for example on a screen for an operator to use in controlling the plant or to an aeration control system 220 to automatically control the aeration such that it is sufficient, but not significantly in excess of that required given the amount of food/biomass the plant is coping with. This in turn enables the plant to operate efficiently but also to react to shock loads and variations in food/biomass levels over time periods of one or more days, weeks, months or years.

In embodiments, as described later, optionally the food-to-biomass ratio and/or BOD (biochemical oxygen demand) data is provided for different classes of organism, potentially at different monitoring locations, for example carboniferous and nitrogenous organisms.

We have previously described a system for monitoring the metabolism/growth of microorganisms, the system comprising a sealed chamber with a flexible diaphragm to provide sensitive pressure measurements of gas pressure in the headspace above a culture liquid. For details reference may be made, for example, to our U.S. Pat. No. 8,389,274.

It is helpful to outline details of such a device since a similar pressure measuring system may be adapted for inclusion in embodiments of the invention described later. Thus FIGS. 2a and 2b show, schematically, an embodiment of a similar device 100 to that in U.S. Pat. No. 8,389,274 under, respectively, normal atmospheric pressure and negative pressure (in operation either negative pressure or positive pressure may be produced). Thus a culture 102 of biological material undergoes metabolism and growth during which it exchanges gases with the aqueous liquid (water) carrying cells depending upon various factors gas may be used and/or produced, for example the cells may produce carbon dioxide during respiration. A gaseous headspace 104 of the sealed culture chamber 106 thus experiences changes in pressure due to exchange of gas with the culture medium, and these are monitored by a diaphragm 108 and converted to an electronic pressure signal 110 which may, for example, be digitised and processed electronically by hardware, software or a combination of the two. As illustrated the device includes a sealable inlet/outlet port 114; it also includes an agitator 112, and may incorporate temperature control (not shown). The liquid phase (sample) to gaseous phase (measured head space) volume ratio can be used to adjust the sensitivity of the device—for example a ratio of up to 1:1 liquid : gas may be employed.

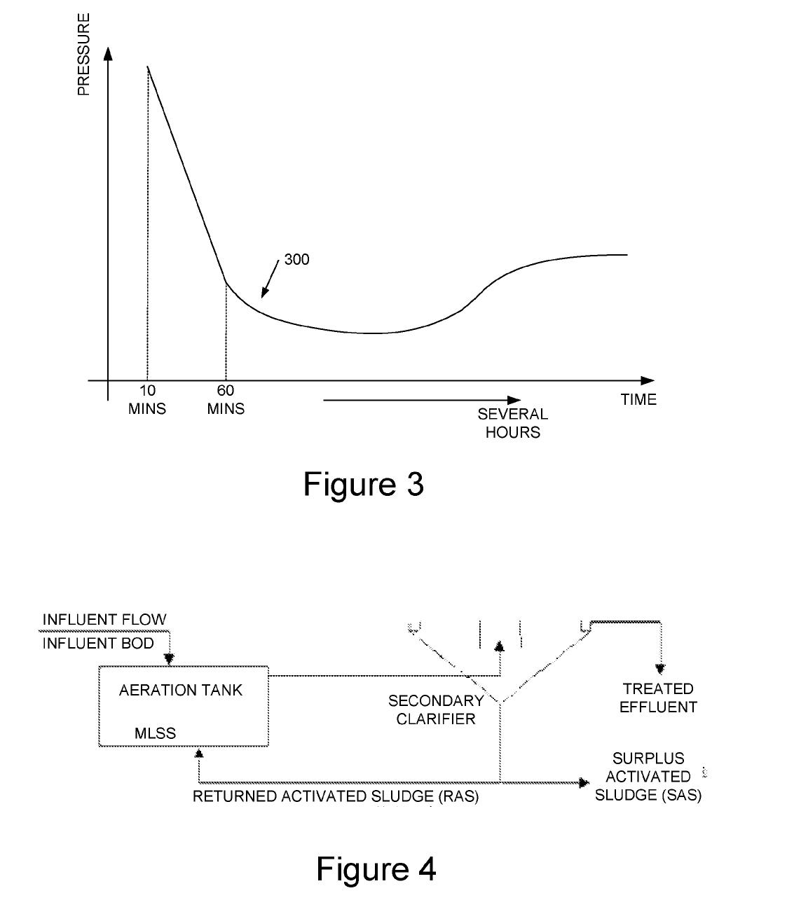

FIG. 3 shows the general shape of a pressure-time curve for a sample of liquid from a sewage treatment plant. Thus there is an initial period during which the pressure can vary and results appear unreliable. This typically lasts up to around 10 minutes. The pressure then begins to fall, flattening out in a trough region 300 after around an hour. Over a further period of several hours the pressure then gradually starts to rise once more (the graph of FIG. 3 is not to scale). The initial rate of pressure drop appears to be related to the concentration of food present, a faster drop being observed with more “food” present. (Thus either the pressure drop or the rate of pressure drop may be measured). Without wishing to be bound by theory it is surmised that the pressure drop relates to the conversion of gas into living biomass and that the trough region occurs when the oxygen has been depleted (the subsequent smaller rise relating to anaerobic respiration). In practice the pressure drop may be a measurement of both BOD and COD (chemical oxygen demand)—but if so this is potentially advantageous for aeration control.

In embodiments this approach provides a “BOD5” test proxy. More particularly the area under the pressure-time curve to this point may also be used as an indication of the amount of food available, and in embodiments may provide a better proxy for a BOD5 test.

Thus, broadly speaking, a closed vessel pressure measurement can be used as a measure of oxygen utilisation by a given body of biomass with time, consistent with the food availability. Additionally or alternatively it can be useful to control based on a food to biomass ratio. If necessary a measurement of the biomass may either be made by heating a sample, for example by microwaving the sample, to determine the dry weight of biomass or by measuring the amount of biomass indirectly by culturing the biomass.

### Food-to-Biomass Ratio Measurement

The inventors have correlated MLSS to Permittivity/Capacitance and have thus been able to characterise MLSS in real-time on-line. More particularly the volatile or viable MLSS is measured because this only sees the live cells and not dead cells and other organic and inorganic matter that makes up MLSS. This facilitates advising on the amount of Return Activated Sludge required to seed the process at any point in time, and doing this dynamically.

In addition to the techniques we describe herein, this is also important because in a toxic event where cells are being killed, or in poorly operating plant, the MLSS may have only a small concentration of live cells and thus conventional dry weight methods of measuring the MLSS will fail (sampling a volume from the AS lane, filtering it to concentrate the solids, then drying).

An important control parameter for the activated sludge process of a sewage treatment plant is the relationship between the load (this may be measured in kg/day) of BOD or bacterial ‘food’ entering the aeration plant, and the ‘mass’ of bacteria in the aeration tank available to treat the incoming BOD. This is referred to as the Food to (bio)Mass Ratio (F:M ratio), sometimes also referred to as the Sludge Loading Rate (SLR). The F:M ratio is perhaps the single most important parameter in controlling the activated sludge process; it may notionally be defined as the kg of BOD5 applied per kg MLSS per day.

The amount of biomass within the reactor is referred to as the Mixed Liquor Suspended Solids (MLSS) (mixed liquor combines raw/unsettled waste water and activated sludge). One approach to establish the MLSS is by filtration and drying at 105° C. to constant weight; it is then possible to calculate the F:M ratio using the model shown in FIG. 4.

Referring now to FIG. 4, Food to (bio)Mass Ratio according to this model may be calculated as follows:

FB:M=[(Bi×Qi)/SMLx Va)]×10⁻³×24

where
Influent flow=Qi (m³/hr)
Influent BOD=Bi (mg/l)
Aeration tank volume=Va (m³)

MLSS=SML (g/l)

and where, in this example, FB:M refers to the loading measured by the BOD5 technique.

In embodiments of the techniques we describe the volatile or viable MLSS is linked to to a measure of biomass activity as measured by oxygen uptake rate or similar measurement, which is representative of food availability, in particular where there are more bacteria than food for them, so that the respiration measured represents the available food. In effect biomass activity is linked with biomass live mass taking into account only the active constituents that process the effluent. Thus we can accurately calculate (automatically and dynamically) the F:M ratio on-line in near real-time. This facilitates a high degree of process efficiency, and substantial reductions in costs, energy input, and carbon emissions.

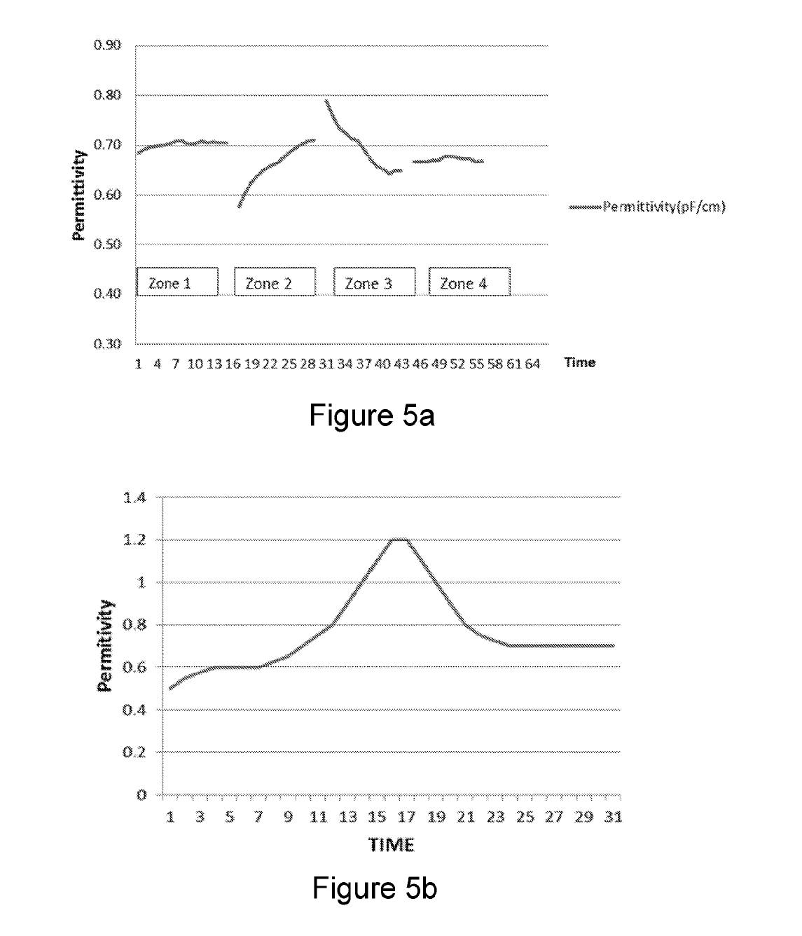

FIG. 5a shows the measured growth of biomass in four zones of an activated sludge (AS) lane of a waste water treatment plant, where permittivity is a proxy for the number of viable organisms. In FIG. 5a an increase in permittivity reflects an increase in growth in four consecutive zones of a plug flow activated sludge lane. Each zone has its own growth characteristics for that particular time point (flow, food availability and the like). As the example illustrates, the inventors' measurements have shown that biomass growth can be determined in zones along the Activated Sludge lane, demonstrating that the lane can be “mapped” to show how the process is working. This may be modelled in a controlled culture vessel to mimic the activated sludge process over time to its conclusion. This is useful as it indicates the overall biological process duration for the particular food/flow characteristics at that time. In general the growth of organisms follows a particular pattern for the given plant conditions; FIG. 5b shows an example of this.

A change in the conditions such as food input (influent concentration) or flow rate will in general affect the profile of the “map”. For example:
As flow rate and food concentration changes, the position of peak biomass concentration changes in relation to time
As flow rate and food concentration changes, the height of peak biomass changes
As flow rate and food concentration changes, the slope of permittivity changes in growth and decline phases

These parameters can be used to further optimise the treatment process in near-real time.

### Treatment Plants and Control

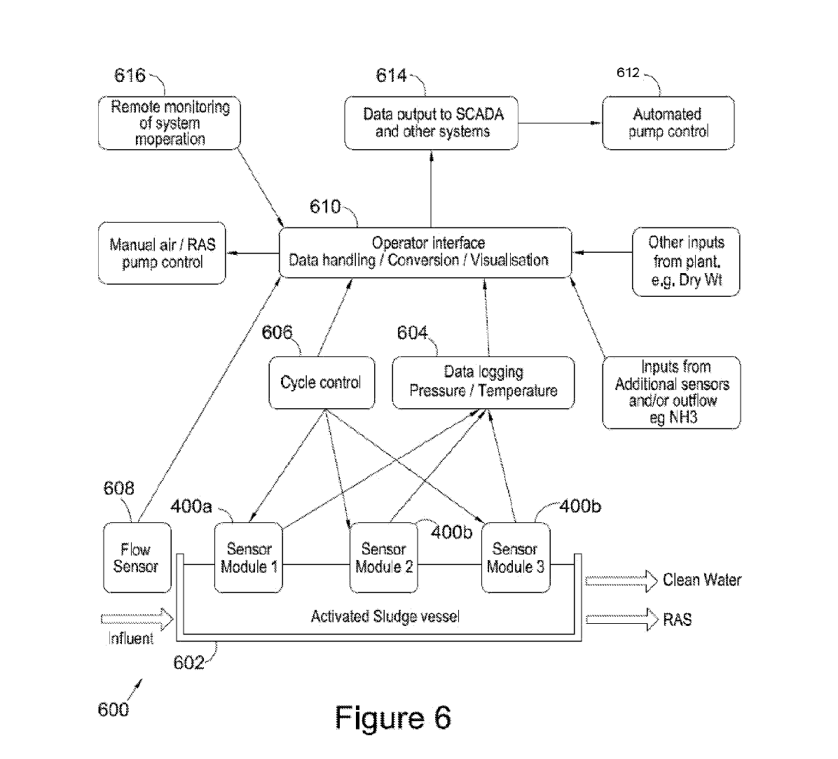

FIG. 6 shows an embodiment of a sewage treatment plant control system 600, illustrating a system of the type shown in FIG. 1 in more detail. Thus an activated sludge vessel 602 is provided (in this example) with three food-to-biomass sensor modules 400a, b, c each coupled to a data logging system 604. In embodiments each sensor module comprises a respirometer and capacitance/permittivity measuring apparatus as previously described.

In embodiments a sensor module may also include a temperature measuring device to provide fluid temperature data back to data logger 604. An optional controller 606 interfaces with and controls the sensor modules. A flow sensor 608 measures a rate of liquid flow into and/or within activated sludge vessel 602. A data handling and visualisation system 610 is connected to the data logging system 604 to receive data from the sensor(s), to controller 606, to control when measurements are made, and to flow sensor 608. The data handling system 610 may thus receive liquid flow data and/or temperature data and/or pressure or gaseous composition measurement data from the one or more sensor modules. The data handling system 610 may present this as raw data to the operator, for example on a graphical display and/or this data may be processed, for example to convert a measurement of gaseous pressure/composition to an indication of oxygen demand and/or an indication of a need for aeration; again one or more of these may optionally be displayed graphically or output in some other manner by module 610. In general module 610 also provides an operator interface to allow control of the sensing modules to make measurements. Optionally module 610 may also receive inputs from one or more additional sensors such as an output flow rate sensor, and/or an ammonium level sensor, and the like. Module 610 may further optionally receive additional inputs from the plant, for example an input of dry biomass weight obtained from drying a sample from one or more locations in the vessel.

In embodiments the information output by module 610 may be employed by an operator of the plant for manual control of a level of aeration and/or for control of a flow rate of sludge through vessel 602 (by controlling a pump), and/or for controlling a degree of RAS feedback (by controlling a RAS pump). In a typical activated sludge vessel aeration may be provided by a series of tubes with holes at intervals along their length provided with an air supply and located at the bottom of the sludge vessel; these tubes may run perpendicular to the flow direction and it may be possible to control aeration so that at different locations along the flow different levels of aeration are provided. Thus the data from module 610 may be employed to control a degree of local aeration, for example in the region of a particular sensor.

Additionally or alternatively the plant may include a control module 612 as part of a system for automatic control of aeration/local aeration and/or of sludge flow rate and/or of RAS feedback. Optionally this control may be implemented by means of an SCADA (supervisory control and data acquisition) interface module 614. Further optionally a network connection/interface 616 may be provided for remote monitoring and/or control of the system. The skilled person will appreciate that the modules 604, 606, 610, 614 and 616 may be implemented as software modules within a computer system; the air/sludge pump control module 612 may be implemented by software with an interface to a suitable electronic controller.

In an automatic arrangement broadly speaking the system may increase a level of aeration when the oxygen demand is high as indicated by a larger measured pressure drop and vice versa. The operating region of the plant may be controlled to be different at different points along the length of flow through vessel 602—for example a region of relatively reduced oxygenation may be provided at the front end of the vessel (where the influent enters) and, for example, a quantity of nitrifying organisms may be controlled so that there is a region of increased nitrification towards an end of the flow region. The skilled person will appreciate that although vessel 602 is illustratively shown as a single vessel; in practice it may comprise multiple linked tanks.

### Selective Sensing

The approaches we have previously described enable the identification of changes in the microbial population over time, or throughout the process, along the lane and/or elsewhere, and may be enhanced by using a range of scanning frequencies. In single species cultures, there is a characteristic frequency profile that is optimal for measuring that organism. When using a range of scanning frequencies there are changes in the profile of a scan when the mixed population changes. This is useful because there is more than one microbiological process going on at the same time in the Activated Sludge lane. Thus an ability to visualise these different processes can be highly advantageous.

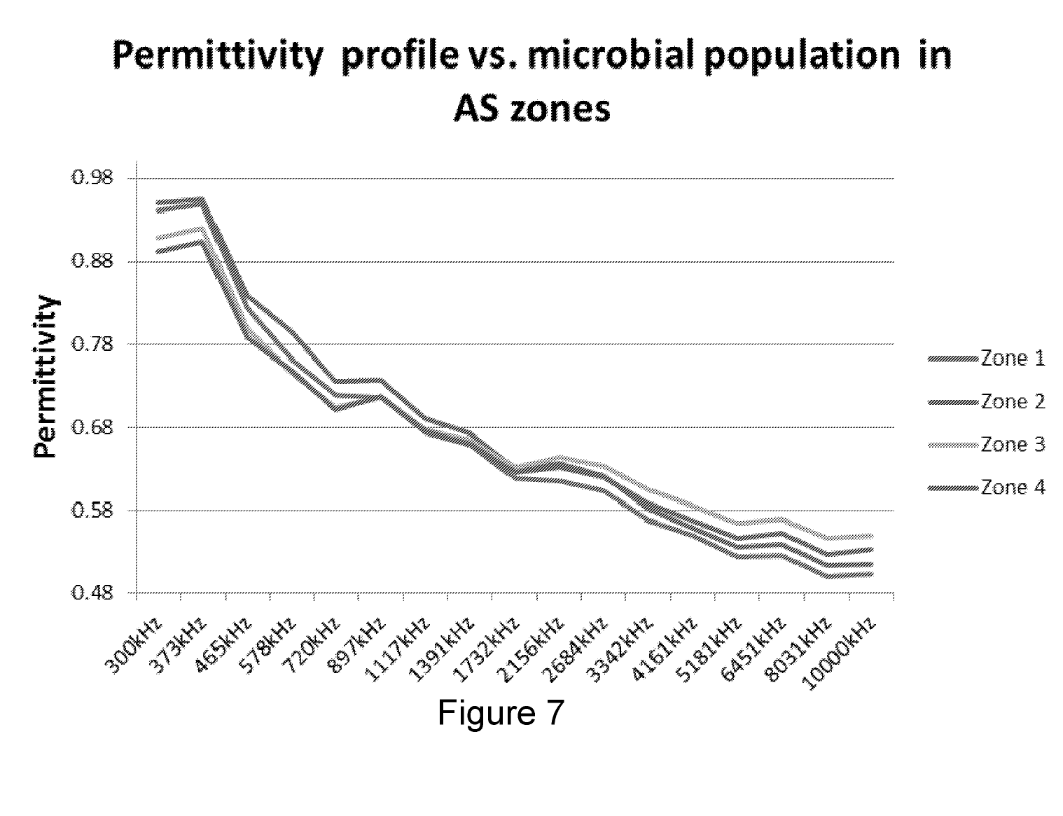

Referring now to FIG. 7, this illustrates a measurement of the electrical permittivity four different zones along an AS lane of a waste water treatment plant. The graph illustrates the central, sloping portion of an S-curve—the permittivity levels out to the left and right of the ends of the illustrated parts.

Each zone has its own permittivity profile, and this changes from one end of the process to the other (over zones 1-4). This is significant, indicating the differences between, for example, early Carbonaceous breakdown and secondary Nitrogenous processing by different microbial species. The ability to visualise the growth of these organisms in their respective process positions is useful in being able to model and/or run a plant efficiently

More particularly, the central sloping parts of the curves of FIG. 7 are relatively gently sloping; with a substantially homogeneous culture of organisms the transition between relatively higher and lower permittivity is much sharper. The gentle slope is attributed to the mixture of types of organism present. Although not easy to distinguish in FIG. 7, the sloping parts of the curves also exhibit various features which depart from a smooth curve; these are believed to be representative of the different types of organism which were present in the samples.

### Rate of Viable Biomass Growth

An additional measure of the health and growth of the biomass under given conditions within a plant is the rate of growth of the viable biomass (for example division and increased cell membrane area) for the viable biomass in relation to the prevailing conditions, e.g. available food and gasses (such as oxygen).

Advantageously, this may provide an additional set of data that provides assurance that aeration/RAS control loops are only affecting the biomass in a positive way.

The method, in particular, comprises determining a rate of growth of the viable biomass by determining a plurality of measures of viable biomass in the aqueous fluid over a first period of time, and then calculating a rate of change of the measure of viable biomass over that first period of time. These measures provide the ability to calculate, in real time, a rate of change of the viable biomass, which gives a measure of the rate of growth of the viable biomass.

The measures of the viable biomass are carried out using the permittivity techniques described above, which provide a measure of the amount of viable biomass in the aqueous fluid.

The rate of growth of the viable biomass may then be used either to control the degree of aeration and/or a volume of food in a water treatment plant, for example.

As described above, it may also be possible to measure the rate of growth for specific groups of organisms by use of certain permittivity frequencies (as outlined above) therefore determining which groups are reacting to deal with a given food source.

Furthermore, this method may be used in conjunction with the above methods of determining a food-to-biomass ratio in the aqueous fluid. Alternatively, the method of determining the rate of growth of the viable biomass may be operated in isolation, that is, without the need to determine the food-to-biomass ratio.

### Rate of Conversion of Food to Biomass

There are known calculations for the average conversion of food into new biomass for wastewater plants. However, these known techniques do not offer real-time calculations, which may be used to fine tune the system. By looking at the growth of organisms and when this peaks a determination can be made of when all the food is converted. The rate of conversion may for example be indicative of the type, or changing type of food source, or how ideal the conditions are for bugs to convert it, how long the conversion of the entire food source will take (big energy implications), or how adaptable the microflora is in dealing with changing food source.

By using the techniques we describe, the rate of conversion of food to biomass is provided over a time period, which provides a measure of this conversion factor under any given plant condition and can therefore be used to fine tune the system.

The method, in particular, comprises: determining a plurality of measures of an amount of food in the aqueous fluid available to the biomass over a period of time, which is indicative of a rate of change of amount of food over the period of time, and then determining a plurality of measures of viable biomass in the aqueous fluid over the period of time, which are indicative of a rate of change of viable biomass over the period of time. From these determined measurements, a calculation of a rate of conversion of food to viable biomass can be made by comparing the rate of change of food and rate of change of biomass over the period of time.

The measures of the viable biomass are carried out using the permittivity techniques described above, which provide a measure of the amount of viable biomass in the aqueous fluid.

The rate of conversion of food to biomass may then be used either to control the degree of aeration and/or a volume of food in a water treatment plant, for example.

As described above, it may also be possible to measure the rate of conversion for specific groups of organisms by use of certain permittivity frequencies (as outlined above) therefore determining which groups are reacting to deal with a given food source.

Furthermore, this method may be used in conjunction with the above methods of determining a food-to-biomass ratio in the aqueous fluid (and also with the method of determining the rate of growth of biomass). Alternatively, the method of determining the rate of conversion of food to viable biomass may be operated in isolation, that is, without the need to determine the food-to-biomass ratio or the rate of growth of viable biomass.

### Alternative Sensors

The above examples describe systems and methods based on the amount of food present in the aqueous fluid being detected by a respirometer, which provides a level of respiration of the viable mass, which is indicative of the amount of viable biomass. In alternative embodiments, the sensor may instead be a sensor of the type for detecting one or more of ammonia, ammonium, nitrites or nitrates. In such embodiments, the ammonia, ammonium, nitrite or nitrate sensor replaces the respirometer, and a measure of the amount of one or more of ammonia, ammonium, nitrite or nitrate is provided, which are indicative of the amount of food present in the aqueous fluid.

Example sensors include optical sensors and ion sensors.

### Example Installations

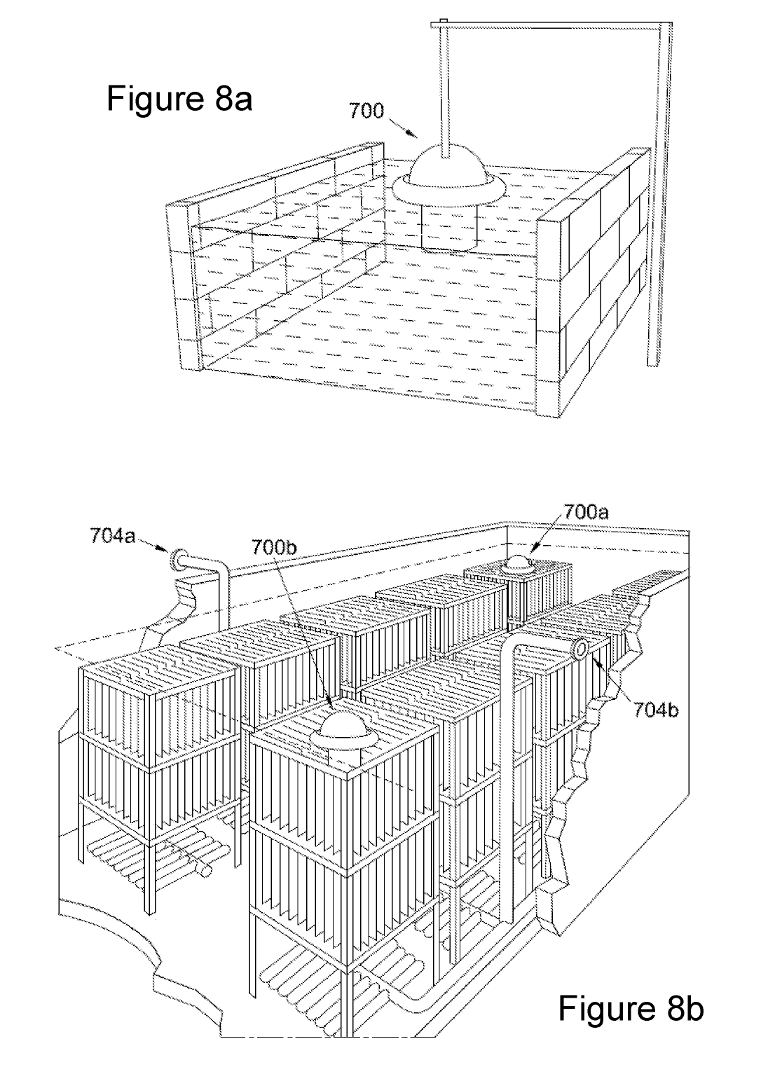

FIG. 8a illustrates a tethered sensing system 700 of the type we have previously described. In some installations there may be two sensors, one at the start and one at the end of the treatment process (and more sensors may be used). Monitoring at multiple points in a waste water treatment process enables different levels of aeration to be employed at the different locations, thus giving rise to energy savings. Thus in embodiments a waste water treatment plant may segregate treatment sections along the flow path providing separate oxygen requirement sensing and aeration control for each section. This has the potential to result in substantial energy savings. Thus FIG. 8b illustrates the use a pair of sensing systems 700a, b, each monitoring a region of immobilised biomass (using curtains) with its own respective aeration 704a, b.

A food-to-biomass sensing system of the type we have described may potentially be employed to monitor toxicity of waste water either in a sewage treatment works or in other industrial plant, or potentially in the outfall from an industrial plant. Although the food-to-biomass sensing system we have described is particularly useful in monitoring a sewage treatment plant it may, more generally, be employed to monitor other water-based processes, for example water in a hospital, water in an air-conditioning system or the like.

No doubt many other effective alternatives will occur to the skilled person. It will be understood that the invention is not limited to the described embodiments and encompasses modifications apparent to those skilled in the art lying within the spirit and scope of the claims appended hereto.

1. A method of determining a food-to-biomass ratio in an aqueous fluid the method comprising:
providing an aqueous fluid comprising viable biomass and food for said biomass, and wherein there is insufficient available food to sustain all said viable biomass;
using a sensor to determine an amount of food in said aqueous fluid available to said biomass;
determining a measure of viable biomass in said aqueous fluid by measuring polarisability of viable biomass cells in an AC electric field; and
determining a food-to-biomass ratio from said amount of food and said measure of viable biomass.

2. A method as claimed in claim 1, wherein said sensor is a respirometer for determining a level of respiration of biomass within said aqueous fluid, the level of respiration representing an amount of food in said aqueous fluid available to said biomass.

3. A method as claimed in claim 2 wherein said food-to-biomass ratio is determined from a ratio of first and second values dependent on said level of respiration and on said measure of viable biomass respectively.

4. A method as claimed in claim 1, wherein said sensor is an ammonia sensor, an ammonium sensor, nitrite sensor or a nitrate sensor for determining a level of ammonia, ammonium, nitrites or nitrates in said aqueous fluid that is indicative of an amount of food in said aqueous fluid available to said biomass.

5. A method as claimed in claim 4 wherein said food-to-biomass ratio is determined from a ratio of first and second values dependent on said level of ammonia, ammonium, nitrites or nitrates and on said measure of viable biomass respectively.

6. A method as claimed in claim 1 wherein said determining of said measure of viable biomass comprises measuring a real or complex electrical permittivity of said aqueous fluid to measure said polarisability.

7. A method as claimed in claim 1 wherein said determining of said measure of viable biomass comprises measuring said polarisability at a plurality of different AC frequencies.

8. A method as claimed in claim 1 wherein said aqueous fluid comprises sewage sludge.

9. A method as claimed in claim 1, wherein said aqueous fluid comprises sewage sludge of a waste water treatment plant, the method further comprising:
determining a food-to-biomass ratio of said aqueous fluid of said waste water treatment plant by monitoring said aqueous fluid in-situ in said plant; and
controlling a degree of aeration of said plant responsive to said determined food-to-biomass ratio.

10. A method as claimed in claim 1 further comprising differentiating between different cell types within said biomass such that said measure of viable biomass comprises a selective measure of a selected said cell type, and wherein said determined food-to-biomass ratio comprises selective food-to-biomass ratio for a selected subset of said viable biomass in said aqueous fluid.

11. A method as claimed in claim 10 wherein said aqueous fluid comprises sewage sludge of a waste water treatment plant, the method further comprising:
determining a food-to-biomass ratio of said aqueous fluid of said waste water treatment plant by monitoring said aqueous fluid in-situ in said plant; and
controlling a degree of aeration of said plant responsive to said determined food-to-biomass ratio;
wherein a first selected said cell type comprises a carbonaceous cell type and said selective food-to-biomass ratio comprises a first selective food-to-biomass ratio more or preferentially responsive to viable carbonaceous cell biomass than to viable nitrogenous cell biomass.

12. A method as claimed in claim 10 wherein said aqueous fluid comprises sewage sludge of a waste water treatment plant, the method further comprising:
determining a food-to-biomass ratio of said aqueous fluid of said waste water treatment plant by monitoring said aqueous fluid in-situ in said plant; and
controlling a degree of aeration of said plant responsive to said determined food-to-biomass ratio;
wherein a second selected cell type comprises a nitrogenous cell type, and said selective food-to-biomass ratio comprises a second selective food-to-biomass ratio more responsive to viable nitrogenous cell biomass than to viable carbonaceous cell biomass.

13. A method as claimed in claim 10 wherein a first selected said cell type comprises a carbonaceous cell type and said selective food-to-biomass ratio comprises a first selective food-to-biomass ratio more responsive to viable carbonaceous cell biomass than to viable nitrogenous cell biomass;
the method further comprising:
determining first and second selective food to biomass ratios of said aqueous fluid of said waste water treatment plant by monitoring said aqueous fluid in-situ in said plant; and
controlling said waste water treatment plant responsive to both said first and second selective food-to-biomass ratios.

14. A method as claimed in claim 1, further comprising determining a rate of growth of said viable biomass by determining a plurality of measures of viable biomass in said aqueous fluid over a first period of time, and calculating a rate of change of said measure of viable biomass over said first period, said rate of change of said measures of viable biomass being indicative of a rate of growth of said viable biomass.

15-17. (canceled)

18. A method as claimed in claim 1, further comprising determining a rate of conversion of food to viable biomass by:
determining a plurality of measures of an amount of food in said aqueous fluid available to said biomass over a second period indicative of a rate of change of amount of food over said second period of time; and
determining a plurality of measures of viable biomass in said aqueous fluid over said second period of time indicative of a rate of change of viable biomass over said second period; and
calculating a rate of conversion of food to viable biomass by comparing the rate of change of food and rate of change of biomass over said second period of time.

19-21. (canceled)

22. A system for determining a food-to-biomass ratio in an aqueous fluid, the system comprising:
a sensor to determine an amount of food in said aqueous fluid available to said biomass;
cell polarisability measuring apparatus to determine a measure of viable biomass in said aqueous fluid by measuring polarisability of viable biomass cells in an AC electric field; and
a system to determine a food-to-biomass ratio from said amount of food and said measure of viable biomass.

23. A system as claimed in claim 22, wherein said sensor is a respirometer for determining a level of respiration of biomass within said aqueous fluid, the level of respiration representing an amount of food in said aqueous fluid available to said biomass.

24. A system as claimed in claim 22, wherein said sensor is an ammonia sensor, an ammonium sensor, nitrite sensor or a nitrate sensor for determining a level of ammonia, ammonium, nitrites or nitrates in said aqueous fluid that is indicative of an amount of food in said aqueous fluid available to said biomass.

25-27. (canceled)

28. A system for differentiating between, or providing a selective response to, different types or classes of viable biomass in activated sludge (AS), the system comprising:
a probe for electrically probing said activated sludge (AS); and
cell polarisability measuring apparatus, couplable to said probe, to determine a measure of viable biomass in said aqueous fluid by measuring polarisability of viable biomass cells in an AC electric field;
wherein said apparatus is configured to differentiate between said different types or classes of viable biomass organisms by operating at one or more selected frequencies or frequency ranges of said AC electric field.

29. (canceled)
